(19) United States
(12) Patent Application Publication
(10) Pub. No.: US 2021/0154108 A1
(43) Pub. Date: May 27, 2021

(54) MAGNETIC COMPOSITION COMPRISING A POLYMER BLEND AND HIGH LOADING OF MAGNETIC MATERIAL

(71) Applicant: L'OREAL, Paris (FR)

(72) Inventors: Xian Zhi ZHOU, Millburn, NJ (US); Christopher PANG, New York, NY (US)

(73) Assignee: L'OREAL, PARIS (FR)

(21) Appl. No.: 16/697,856

(22) Filed: Nov. 27, 2019

(51) Int. Cl. A61K 8/19; A61K 8/81; A61Q 1/10

(52) U.S. Cl. CPC A61K 8/19; A61K 8/8152; A61Q 1/10; A61K 2800/30; A61K 2800/47; A61K 2800/621; A61K 8/8117

(57) ABSTRACT

An eyeliner composition includes water, a styrenated polymer, an unstyrenated polymer, and a surface-treated, magnetic, iron-containing particulate. Methods applying a magnetic false eyelash using the eyeliner composition are also provided.

### FIELD OF THE INVENTION

The present invention relates to a cosmetic composition for keratinous materials such as the eyelid. The cosmetic composition is preferably a magnetic eyeliner.

### DISCUSSION OF THE BACKGROUND

Eyeliner compositions are commonly used to enhance the appearance of a user's eyes. Eyeliners that include magnetic materials have been taught for use to adhere a magnetic false eyelash to a user's eyelid. However, the inventors have found that conventional magnetic eyeliners do not combine strong magnetic as well as physical/chemical adhesive holding power in combination with other desirable features such as easy application, comfortable wear, limited flaking or smudging, and/or easy removability.

The inventors of the present invention have found that certain eyeliner formulations with styrenated polymer and unstyrenated polymer are useful for obviating one or more of the above-mentioned drawbacks.

### SUMMARY OF THE INVENTION

According to certain aspects of the present invention, an eyeliner composition includes water; a styrenated acrylic polymer; an unstyrenated acrylic polymer; and at least about 40% by weight of a surface-treated, magnetic, iron-containing particulate.

According to other aspects of the present invention, a method of applying a magnetic false eyelash is provided. The method includes applying an eyeliner composition to a surface of a user's eyelid. The eyeliner composition is capable of fixing the magnetic false eyelash to the user's eyelid. The eyeliner composition includes a styrenated polymer, an unstyrenated polymer; and at least about 25% by weight of a surface treated, magnetic iron-containing particulate.

It is to be understood that both the foregoing general description and the following detailed description are exemplary and explanatory only, and are not restrictive of the invention.

### DETAILED DESCRIPTION OF THE INVENTION

Unless otherwise indicated, all numbers expressing quantities of ingredients and/or reaction conditions are to be understood as being modified in all instances by the term “about.” Accordingly, unless indicated to the contrary, the numerical parameters set forth in the following specification and attached claims are approximations that may vary depending upon the desired properties sought to be obtained by the present invention.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number. For example, about 10% means from 9% to 11% or 8.5% to 11.5%.

As used herein, “eyeliner” and “eyeliner composition” mean a composition that is intended to be applied to keratinous materials, in particular eyelids, more particularly the portion of the eyelid adjoining the eyelashes.

“Percent” or “%” as used herein, when referring to concentrations of ingredients or components in compositions refers to percent by weight. Unless otherwise specifically stated, the percent of a particular ingredient or ingredients is on an actives basis and unless otherwise specifically stated is relative to the entire composition.

As used herein, all ranges provided are meant to include every specific range within, and combination of sub ranges between, the given ranges. Furthermore, notably the range description “from about 1%, 2% or 3% to about 5%, 10% or 15%,” includes 1%-5%, 1%-10%, %-15%, 2%-5%, 2%-10%, 2%-15%, 3%-5%, 3%-10%, and 3%-15%.

“Actives basis” refers to the amount of a particular ingredient exclusive of any solvents, carriers, impurities and the like that may be supplied with the particular ingredient.

“Substantially free” as used herein to refer to the presence of ingredients within compositions of the present invention, means that the particular ingredient is present in concentrations by weight of less than about 1%, such as less than about 0.5%, such as less than about 0.25%, such as about 0%.

“Substituted” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The term “magnetic” refers to materials and compositions having magnetic susceptibility such as that sufficient to impart an attractive magnetic force between a film formed on the skin from eyeliners of the present invention and a false eyelash having small magnetic materials or structures formed therein/thereon.

Composition

According to the present invention, the inventors have found that particular compositions of the present invention have surprising and unexpected properties such as those related to transfer resistance, tack, and rheology. These compositions include a styrenated polymer and a non-styrenated polymer. In certain embodiments the styrenated polymer and the non-styrenated polymer are present in a weight ratio of the styrenated polymer to the non-styrenated polymer that is from about 2:1 to about 5:1. In certain other embodiments the compositions include a surface-treated, magnetic, iron-containing particulate having a coating that includes a hydrophobic titanium material, a silicone material, or combinations thereof.

Polymer Blend

Compositions of the present invention include a polymer blend (a mixture of at least two polymers, where the two polymers have at least one differing repeat unit). In particular, the compositions of the present invention include at least one polymer comprising at least one styrene group (i.e., a styrenated polymer) and at least one polymer lacking styrene groups (i.e., an unstyrenated polymer). In certain embodiments, the styrenated polymer and the unstyrenated polymer are water-dispersible.

According to certain embodiments the styrenated polymer and unstyrenated polymers are film-forming polymers. One of ordinary skill in the art will readily appreciate the term “film-forming polymer” refers or “film forming agent” as used herein means a polymer or resin that leaves a film (e.g., a continuous film) on the substrate to which it is applied, for example, after a solvent accompanying the film former has evaporated, absorbed into and/or dissipated on or from the substrate.

According to certain other embodiments, the styrenated polymer and unstyrenated polymers are latex polymers. By “latex polymers” it is meant polymers emulsion polymerized polymers, in particular, synthetic polymers. According to certain notable embodiments one or both of the styrenated polymer and unstyrenated polymers are acrylic. By “acrylic” it is meant polymers or copolymers having ethylenic unsaturation, e.g., based on the chemistry of acrylic acid. Nonlimiting examples include polymers including monomers that are esters of acrylic acid and metharcylic acid. The monomers may contain any of various functional groups including alkyl, aryl, hydroxyl, amino, amido, and the like. The acrylic polymer may include other co-monomers including (in the case of the styrenated polymer) styrenated monomers.

In certain embodiments, the styrenated polymer is a styrenated acrylate such as STYRENE/ACRYLATES/AMMONIUM METHACRYLATE COPOLYMER and may be a commercially available variety, such as SYNTRAN 5760, commercially available from Interpolymer Corporation of Canton, Mass.

The concentration of the styrenated polymer in the composition may be from about 6%, 8%, 9% or 10% to about 10%, 15%, 20% or 25%.

In certain embodiments, the unstyrenated polymer is a copolymer comprising two or more monomers chosen from acrylic acid, methacrylic acid, and their simple esters, for example, lower alkyl esters such as methyl, ethyl, and ethylhexyl esters. For example, copolymers may be chosen from ammonium acrylates copolymers, ethyl acrylates copolymers, acrylates/ethylhexylacrylate copolymers, acrylates/octylacrylates copolymers, alkyl (meth)acrylates copolymers, acrylates/C₁₂-C₂₂ alkylmethacrylate copolymers, ethylacrylate/methacrylic acid copolymer, ethylhexyl acrylate/butyl acrylate/methacrylic acid, and t-butyl acrylate/ethyl acrylate/methacrylic acid copolymer. Examples of commercially available acrylate copolymers include, but are not limited to, DAITOSOL 3000 SLPN, DAITOSOL 4000 SJT, DAITOSOL 5000 AD, DAITOSOL 5000 SJ, commercially available from Daito Kasei Kogyo Corporation of Osaka, Japan. According to certain embodiments, the unstyrenated polymer includes at least one monomer which is acrylic acid or methacrylic acid.

The concentration of the unstyrenated polymer in the composition may be from about 2%, 3%, or 5% to about 5%, 10%, or 15%.

The inventors have found that the total (actives basis) concentration of styrenated polymer plus unstyrenated polymer (and, in particular styrenated acrylic and unstyrenated acrylic) is desirably between about 10% or 13% to about 20% or 25% by weight in order to provide sufficient but not too much viscosity building as well as to provide sufficient tackifying (physical-chemical) force to aid in holding a magnetic lash to an eyeliner film.

Furthermore, surprisingly, the inventors have also found that in order to additionally maintain the viscosity and tack described above and additionally provide strong transfer resistance, the ratio (by weight concentration of active polymer) of the concentration of styrenated polymer to that of the unstyrenated polymer (and, in particularly notable embodiments, the concentration of styrenated acrylic to unstyrenated acrylic) is desirably held between about 2:1 and about 5:1. Stated differently, in this embodiment, the concentration by weight of styrenated polymer is from about two to five times that of the unstyrenated polymer.

In certain embodiments additional polymers are included in the composition. For example, film-forming polymers such as cellulose polymers may be included. In other embodiments thickening polymers such as xanthan or other natural gums or sodium polyacrylate are included. In certain notable embodiments, the polymers in the composition are limited to the one or more styrenated polymers, the one or more unstyrenated acrylic polymers, and less than about 1% by weight of such additional polymers (which are neither styrenated acrylic nor unstyrenated acrylic; as such are lacking in ethylenic unsaturation). In certain embodiments the compositions include from about 0.1% or 0.25% to about 0.5% or 1% of additional polymers, such as those selected from (unstyrenated and lacking in ethylenic unsaturation) natural or synthetic gums or cellulose polymers.

Magnetic, Iron-Containing Particulate

The composition also contains a magnetic iron-containing particulate material such as a colorant that has magnetic properties, which may be a magnetic or paramagnetic material. Particularly suitable materials are those comprising iron oxide Fe₃O₄. By way of example, pigments presenting magnetic properties are those black iron oxide particles, e.g., those sold under the trade name SICOVIT noir E172 by BASF, or soft-iron based particles proposed under the trade name STAPA® WM IRON VP 041040 by ECKART, may also be mentioned.

The magnetic iron-containing particulate material may include also comprise metallic iron, in particular passivated soft iron, e.g., obtained from carbonyl iron by implementing the method described in U.S. Pat. No. 6,589,331, the contents of which are incorporated herein by reference. The metallic iron may include a surface oxide layer. According to certain other embodiments, the magnetic material includes iron oxide, iron (metallic), or combinations thereof.

According to certain embodiments the magnetic, iron-containing particulate material comprises at least about 90% by weight of either iron or iron oxide.

According to certain notable embodiments, the magnetic iron-containing particulate material is an iron oxide or iron that has a surface coating that includes a hydrophobic titanium material, a silicone material, or combinations thereof.

Suitable hydrophobic titanium materials include isopropyl titanium triisostearate. Suitable silicone materials include dimethicones as well as organosilanes such as triethoxycaprylylsilane. In certain embodiments the magnetic material is coated with both isopropyl titanium triisostearate and is also coated with a silicone material such as triethoxysilylethyl polydimethylsiloxyethyl dimethicone.

Compositions of the present invention include a high loading of the magnetic material. Accordingly, the magnetic material is present in an amount of at least 25% by weight of the composition. In certain embodiments, the composition includes between 30% and (40% or 50%) by weight of the magnetic material. In preferred embodiments, the composition includes at least about 40% of magnetic material, such as from about 40% to about 50% by weight of the magnetic material. In certain embodiments, the composition includes between about 40% to about 45% magnetic material.

Additional Ingredients

The composition of the present invention may further include various additives desirably used in cosmetic or dermatological compositions. For example, water, humectants, dispersants, anti-oxidants, pH adjusters, preservatives, neutralizing agents, fragrances, fillers, co-solvents, defoamers, cosmetic and dermatological active agents such as emollients, moisturizers, vitamins, UV filters, and sunscreens, and mixtures thereof can be added. A non-exhaustive listing of such ingredients can be found in the CTFA International Cosmetic Ingredient Dictionary and Handbook, Fourteenth Edition (2012), contents of which are incorporated herein by reference in its entirety.

One skilled in the art will take care to select the optional additional additives and/or the amount thereof such that the advantageous properties of the compositions according to the present invention are not, or are not substantially, adversely affected by the envisaged addition.

These substances may be selected variously by one skilled in the art to prepare a composition which has the desired properties, for example, magnetism, tack, viscosity, and/or ease of removability.

According to certain embodiments, compositions of the present invention may include a humectant. By humectant it is meant an ingredient useful at retaining moisture. Suitable humectants may be water soluble such as glycerol or a glycol such as butylene glycol, propylene glycol or hexylene glycol. If present, the concentration of humectant may be from about 0.5% or 1% or 2% to about 4%, 5% or 10% by weight.

In certain embodiments, compositions of the present invention include a cosolvent such as a C₂-C₅ monoalcohol such as ethanol or isopropanol. If present, the concentration of cosolvent may be from about 0.5% or 1% or 2% to about 4%, 5% or 10% by weight.

As used herein, “wax” is intended to mean a lipophilic fatty compound that is solid at room temperature (about 25° C.) and atmospheric pressure (760 mmHg, i.e., 105 Pa), which undergoes a reversible solid/liquid change of state and which has a melting point of greater than 30° C., and in some embodiments, greater than about 55° C. up to about 120° C. or even as high as about 200° C.

The term wax may include waxes of animal origin, waxes of plant origin, waxes of mineral origin and waxes of synthetic origin. Examples of waxes of animal origin include beeswaxes, lanolin waxes and Chinese insect waxes. Examples of waxes of plant origin include rice waxes, carnauba wax, candelilla wax, ouricurry wax, cork fiber waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, e.g., polyethylene waxes, waxes obtained by Fischer-Tropsch synthesis, waxy copolymers and their esters, and silicone and fluoro waxes.

The term wax may further include high melting point hydrogenated oils of animal or plant origin. Examples include carnauba wax, beeswax, polyethylene wax, hydrogenated jojoba waxes and hydrogenated oils which are obtained by catalytic hydrogenation of fats composed of a C₈-C₃₂ linear or nonlinear fatty chain, hydrogenated sunflower oil, hydrogenated castor oil, hydrogenated copra oil, hydrogenated lanolin and hydrogenated palm oils. If present, the amount of the wax in the composition may be from about 0.1, 0.5% or 1% to about 5%, 10% or 20% by weight. However, in certain embodiments the compositions are substantially free of waxes.

Compositions of the present invention may additionally include oils. As used herein, by “oils,” it is meant compounds having a melting point of less than about 30C and generally insoluble in water and includes a hydrophobic moiety, such as one meeting one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. The hydrophobic compound is in certain embodiments not amphiphilic and, as such, in this embodiment does not include hydrophilic moieties, such as anionic, cationic, zwitterionic, or nonionic groups, that are polar, including sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy)sulfonyl moieties. In certain embodiments, the oil does not include hydroxyl moieties.

Suitable examples of compounds of oils include vegetable oils (glyceryl esters of fatty acids, triglycerides) and fatty esters. Specific non-limiting examples include, without limitation, esters such as isopropyl palmitate, isopropyl myristate, isononyl isonanoate C₁₂-C₁₅ alkyl benzoates, caprylic/capric triglycerides, silicone oils (such as dimethicone and cyclopentasiloxane), pentaerythritol tetraoctanoate and mineral oil. Other examples of oils include liquid organic ultraviolet filter commonly used for example as UV-absorbing sunscreens such as octocrylene, octyl salicylate, octyl methoxyxcinnamate, among others. If present, the amount of the oil in the composition may be from about 0.1, 0.5% or 1% to about 5%, 10% or 20% by weight. However, in certain embodiments the compositions are substantially free of oils.

According to certain embodiments of the present invention, the composition may further optionally include an emulsifier, surfactant or dispersant, primarily to assist in wetting or dispersing of the magnetic material or pigments and/or for emulsifying oils or waxes. Any surfactants, including anionic, nonionic, amphoteric, and cationic, surfactants, may be used in the present invention, as long as the surfactant is cosmetically or dermatologically acceptable. The surfactant may be used either singly or in combination two or more thereof. Examples of surfactants/dispersants include potassium cetyl phosphate, steareth 20/2, ceteareth 20, glyceryl stearate/behenate, cetyl alcohol, myristyl alcohol, stearyl alcohol, alkoxylated castor oils, and the like. According to certain embodiments, the surfactant has an HLB value from about 2 to about 10. If present, the amount of the surfactant or dispersant may be from about 0.1 to about 5% by weight.

Additional Colorants and Particulates

The magnetic iron-containing particulate material present in the composition may also serve as colorant. Compositions of the present invention may optionally include at least additional colorant. Suitable additional colorants include, but are not limited to inorganic particulates that impart color or optical effects and organic pigments. Particulate materials are generally finely divided particulates that are insoluble in but are otherwise homogeneously stabilized (suspended or dispersed) in a vehicle of the composition. The one or more particulate materials are typically materials that are incapable of chemically “self-fusing” in-use and are not themselves film-forming.

Suitable inorganic particulate materials include any of a variety of porous, semi-porous, non-porous, or hollow, coated or uncoated water-insoluble inorganic particulates such as silica, alumina, carbon and any of various oxides, silicates, aluminosilicates, nitrides, carbides, carbonates, and the like. In particular embodiments, the inorganic particulate is selected from carbon black, silica, and an oxide. If present, the amount of the additional colorant or particulate may be from about 0.1, 0.5% or 1% to about 1%, 10% or 15% by weight.

Vehicle

In order to facilitate application to the eyelid, compositions of the present invention generally include a vehicle in which magnetic material and the styrenated polymer and unstyrenated polymer are stabilized (e.g., dispersed and/or suspended). The vehicle generally includes, consists or consists essentially of water. In certain embodiments of the invention, the compositions of the present invention include at least about 10% of water and/or less than about 50% by weight of water, such as less than about 40% by weight of water, such as less than about 30% by weight of water.

According to certain embodiments, the compositions of the present invention are in the form of a water dispersion where the styrenated polymer, the unstyrenated polymer are dispersed within the water or in the water phase. In other embodiments, the composition may be an emulsion.

According to certain embodiments compositions of the present invention have a viscosity from about 0.1 Pas, 1 Pas or 5 Pas to about 35 Pas, 40 Pas or 50 Pa s including all combinations of such ranges, when measured at a shear rate of 1 s⁻¹ as measured using, for example, the Discover HR-2 TA Instrument rotational rheometer, equipped with an air-cooled Peltier plate to regulate the temperature and a cone and plate geometry 20 mm/angle of 2° and a shear rate stepped from 0.01 to 1000 s-1 at 25° C., available from TA Instruments of New Castle, Del. In order to adjust the viscosity of the formulation, one may use one or more viscosity modifiers. According to certain other embodiments of the invention, the composition may have a pH that is from about 5 to about 8. In certain cases, it the concentration of salts or electrolytes is limited to less than about 2% by weight.

The compositions of the present invention are intended to be applied onto keratinous materials such as eyelids, in particular, on skin adjoining the eyelashes.

As described above, according to one aspect of the present invention, the composition has improved cosmetic properties such as, for example, physical/chemical adhesive holding power in combination with other desirable features such as easy application, comfortable wear, limited flaking or smudging, and/or easy removability, and the like.

Methods

Compositions of the present invention may be made by mixing at least one styrenated polymer and at least one unstyrenated polymer in water (and optional other solvents and other water soluble ingredients) until they are dispersed. The styrenated polymer and unstyrenated polymer and other water-soluble ingredients may be mixed by stirring, shaking, grounding, or beating, optionally with a stirrer, a magnetic stirrer, a shaker, a homogenizer, or any other methods suitably used to mix cosmetic composition. The mixing may be carried out with or without heating or cooling the ingredients. Iron-containing particulates and other optional particulates that are to be dispersed are then added with mixing to form a homogeneous mixture.

One embodiment of the present invention provides a method of applying a magnetic false eyelash, comprising applying to a surface of an eyelid an eyeliner composition (such as in the form of a film) capable of fixing the false eyelash to a user's eyelid. The eyeliner composition described above is applied onto the keratinous materials. The keratinous materials are, in particular on the surface of the eyelid and in particular the skin adjoining the eyelashes. The composition is applied onto the such skin in an amount, for example, sufficient to form a film along the entire surface of the skin that adjoins the user's eyelash. Accordingly, the compositions may be applied by a pencil, wand, brush or similar device.

The compositions may be applied to eyelid and allowed to dry before attaching a magnetic false eyelash thereto. By attaching or “fixing” the magnetic false eyelash to the user's eyelid, it is meant that the magnetic and physical chemical forces between the film of eyeliner and the magnetic eyelash allow the user to align the magnetic false eyelash onto and along the film of magnetic eyeliner.

Notwithstanding that the numerical ranges and parameters setting forth the broad scope of the invention are approximations, the numerical values set forth in the specific examples are reported as precisely as possible. Any numerical value, however, inherently contain errors necessarily resulting from the standard deviation found in their respective measurements.

The inventors have found that eyeliner compositions of the present invention adhere well to magnetic false eyelashes and also have desirable fluidity, tack, and transfer resistance.

### Examples

Nine eyeliner compositions were prepared by combining water, styrenated polymer, unstyrenated polymer, less than 1% of xanthan gum, humectant, preservatives, defoamer as well as 45% of an iron oxide magnetic material. All of the concentrations of ingredients were fixed, but the nature of coating on the iron oxide magnetic material was allowed to vary. The various coatings on the iron oxide are provided in Table 1 below.

Rheological testing was performed on the various compositions using a Discover HR-2 TA Instrument rotational rheometer, equipped with an air-cooled Peltier plate to regulate the temperature. A cone and plate geometry 20 mm/angle 2° was used with an anti-evaporation device to avoid evaporation during measurements. The shear rate was stepped from 0.01 to 1000 s-1 at 25° C.

Surprisingly, the ratio of the nature of the coating on the iron oxide greatly impacts the rheological profile of the composition. The compositions of Examples 1, 2, 3, and 4 each included iron oxide pigment that was coated with hydrophobic titanium material (Isopropyl Titanium Triisostearate), a silicone material (Polydimethylsiloxyethyl Dimethicone or Triethoxycaprylylsilane), or combinations thereof. These were all pourable fluids. This is reflected in their relatively low viscosities. Specifically, these samples had viscosities measured at (1 s⁻¹) of less than 50 Pa S and viscosities measured at (100 s⁻¹) of less than 1 Pa S, as reported in Table 1.

The compositions of Examples 5, 6, 7, 8 and 9 each included iron oxide pigment that was coated with silica or Disodium stearoyl glutamate and aluminum hydroxide or no coating at all. These were much more viscous and either cream-like (Examples 5, 6, 7) or gel-like (Examples 8, 9) and had higher viscosities.

TABLE 1

| | | Particle | Viscosity | Viscosity |
|---|---|---|---|---|
| | | Size Range | @1 s⁻¹ | @100 s⁻¹ |
| Ref. | Coating Material | (microns) | (Pa · S) | (Pa · S) |
| Example 1 | Isopropyl Titanium | 1-3 | 20 | 0.68 |
| | Triisostearate (And) | | | |
| | Triethoxysilylethyl | | | |
| | Polydimethylsiloxy- | | | |
| | ethyl Dimethicone | | | |
| Example 2 | Isopropyl Titanium | 1-5 | 23 | 0.7 |
| | Triisostearate (And) | | | |
| | Triethoxysilylethyl | | | |
| | Polydimethylsiloxy- | | | |
| | ethyl Dimethicone | | | |
| Example 3 | Triethoxycaprylyl- | 0.5-1.1 | 33.9 | 0.98 |
| | silane | | | |
| Example 4 | Isopropyl Titanium | 1-3 | 30.7 | 0.95 |
| | Triisostearate | | | |
| Example 5 | No treatment | 0.3 | 70 | 2.1 |
| Example 6 | Silica | 1-4 | 113 | 2.11 |
| Example 7 | Disodium steroyl | 2-7 | 116 | 3.2 |
| | glutamate and | | | |
| | aluminum hydroxide | | | |
| Example 8 | No treatment | 1-3 | 182 | 8.1 |
| Example 9 | Disodium steroyl | 0.5 | 600 | 15 |
| | glutamate and | | | |
| | aluminum hydroxide | | | |

These results indicate that using high concentrations of magnetic material with a coating that includes a hydrophobic titanium material, a silicone material, or combinations is less susceptible to magnified viscosity. Accordingly using such coatings can make the composition easier to formulate or to apply evenly to the eyelid.

What is claimed is:

1. An eyeliner composition, comprising:
water;
a styrenated acrylic polymer;
an unstyrenated acrylic polymer; and
at least about 40% by weight of a surface-treated, magnetic, iron-containing particulate.

2. The eyeliner composition of claim 1, comprising an additional polymer lacking ethylenic unsaturation, wherein the additional polymer lacking ethylenic unsaturation is a natural gum or cellulose polymer and is present in a concentration by weight from about 0.1% by weight to about 1% by weight.

3. The eyeliner composition of claim 1 wherein the concentration of water is less than about 50% by weight.

4. The eyeliner composition of claim 1 wherein the concentration of water is less than about 40% by weight.

5. The eyeliner composition of claim 1 further comprising a water-soluble humectant.

6. The eyeliner composition of claim 1 wherein the sum of the concentration of styrenated acrylic polymer and the unstyrenated acrylic polymer is from about 13% to about 20% by weight.

7. The eyeliner composition of claim 1 wherein the styrenated acrylic polymer and the unstyrenated acrylic polymer are present in a ratio by weight of styrenated acrylic polymer to unstyrenated acrylic polymer that is from about 2:1 to about 5:1.

8. The eyeliner composition of claim 6 wherein the styrenated acrylic polymer and the unstyrenated acrylic polymer are present in a ratio by weight of styrenated acrylic polymer to unstyrenated acrylic polymer that is from about 2:1 to about 5:1.

9. The eyeliner composition of claim 1 wherein the eyeliner composition is substantially free of oil and wax.

10. The eyeliner composition of claim 1, wherein the surface-treated, magnetic, iron-containing particulate comprises a coating that includes a hydrophobic titanium material, a silicone material, or combinations thereof.

11. A method of applying a magnetic false eyelash, comprising applying to a surface of a user's eyelid an eyeliner composition that is capable of fixing the magnetic false eyelash to the user's eyelid, wherein the eyeliner composition comprises:
water;
a styrenated polymer;
an unstyrenated polymer; and
at least about 25% by weight of a surface-treated, magnetic iron-containing particulate.

12. A method of claim 11 wherein the eyeliner composition comprises at least about 40% by weight of a surface-treated, magnetic, iron-containing particulate.

13. The method of claim 11, wherein the styrenated polymer and the unstyrenated polymer are ethylenically unsaturated.

14. The method of claim 13, wherein the eyeliner composition comprises an additional polymer lacking ethylenic unsaturation, wherein the additional polymer lacking ethylenic unsaturation is present in a concentration by weight of less than about 1% by weight.

15. The method of claim 11 wherein the surface-treated, magnetic, iron-containing particulate comprises a coating that includes a hydrophobic titanium material, a silicone material, or combinations thereof.

16. The method of claim 11 wherein the concentration of water in the eyeliner composition is less than about 40% by weight.

17. The method of claim 13 wherein the sum of the concentration of styrenated polymer and the unstyrenated polymer is from about 13% to about 20% by weight.

18. The method of claim 13 wherein the styrenated polymer and the unstyrenated polymer are present in a ratio by weight of styrenated polymer to unstyrenated polymer that is from about 2:1 to about 5:1.

19. The method of claim 17 wherein the styrenated polymer and the unstyrenated polymer are present in a ratio by weight of styrenated polymer to unstyrenated polymer that is from about 2:1 to about 5:1.

20. The method of claim 11 wherein the eyeliner composition is substantially free of oil and wax.
